(19) United States
(12) Patent Application Publication
(10) Pub. No.: US 2022/0203121 A1
(43) Pub. Date: Jun. 30, 2022

(54) SYSTEM FOR ULTRASOUND TREATMENT COMBINED WITH UVC TREATMENT

(71) Applicants: Innotia Eesti Oü, 10130 Tallinn (EE); OY PACE1 TOOLS LTD, 06100 Porvoo (FI)

(72) Inventors: Mika Lokkinen, Tallinn (EE); Jukka Jokinen, Porvoo (FI)

(21) Appl. No.: 17/438,142

(22) PCT Filed: Apr. 29, 2021
(86) PCT No.: PCT/FI2021/050320

§ 371 Date: Sep. 10, 2021

(30) Foreign Application Priority Data
May 18, 2020 (FI) 20205498

(51) Int. Cl. A61N 5/06; A61N 7/00

(52) U.S. Cl. CPC A61N 5/0624; A61N 2005/0661; A61N 2007/0004; A61N 7/00

(57) ABSTRACT

A treatment system has a base unit and a portable unit. The base unit has a pliable top layer and one or more ultrasonic transducers configured to produce ultrasound waves into the pliable top layer. The portable unit has a pliable pad and an ultrasonic transducer producing ultrasound waves into the pliable pad. The portable unit also has a source of ionizing electromagnetic radiation configured to emit in ionizing electromagnetic radiation.

### FIELD OF THE INVENTION

The invention relates to a treatment system for treating or preventing infections.

### PRIOR ART

Respiratory infections—and infections in general—are caused by viruses and bacteria which mutate over time and may eventually become resistant to antibiotics and other medications. There is a vast number of different viruses and bacteria and the type causing an infection in a patient has to be identified before a proper treatment can be determined. For some viruses and bacteria, there is no treatment available and for some the treatment is not effective enough to save all patients.

### BRIEF DESCRIPTION OF THE INVENTION

The object of the invention is a system, which can target and damage a wide variety of different viruses and bacteria and alleviate infections caused by viruses or bacteria.

Object of the invention is achieved with a two-part system where both units of the system comprise an ultrasonic transducer and at least one unit comprises ultraviolet emitter. The ultrasonic emitters are arranged to emit ultrasound waves into a pliable medium which can be pressed firmly into contact with a patient to achieve efficient propagation of ultrasound waves from the system into the body of the patient.

### BRIEF DESCRIPTION OF THE FIGURES

The invention is now described in more detail in connection with preferred embodiments, with reference to the accompanying drawings, in which:

FIG. 1 shows a treatment system according to an embodiment in use;

FIG. 2 is an isometric view of a base unit according to an embodiment;

FIG. 3 shows a base unit according to an embodiment seen from above;

FIG. 4 is a section view of Section A-A shown in FIG. 3;

FIG. 5 is a section view of Section B-B shown in FIG. 3;

FIG. 6 is an isometric section view of a portable unit according to an embodiment;

FIG. 7 is a section view of Section C-C shown in FIG. 6; and

FIG. 8 shows a portable unit according to an embodiment seen from below;

### DETAILED DESCRIPTION OF THE INVENTION

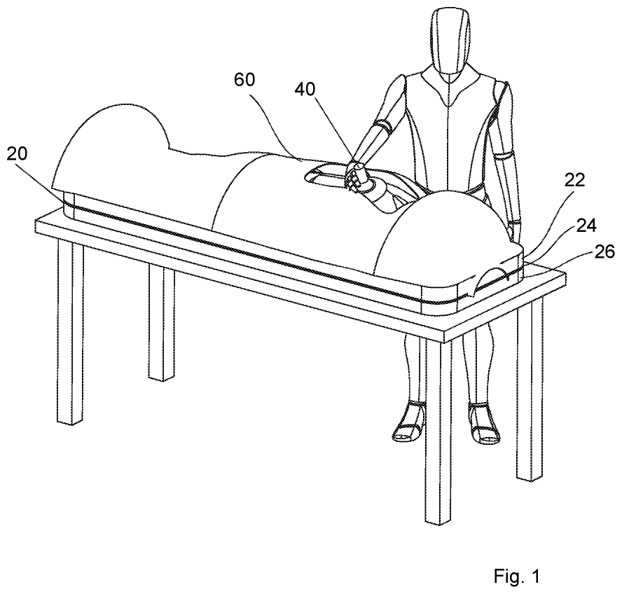

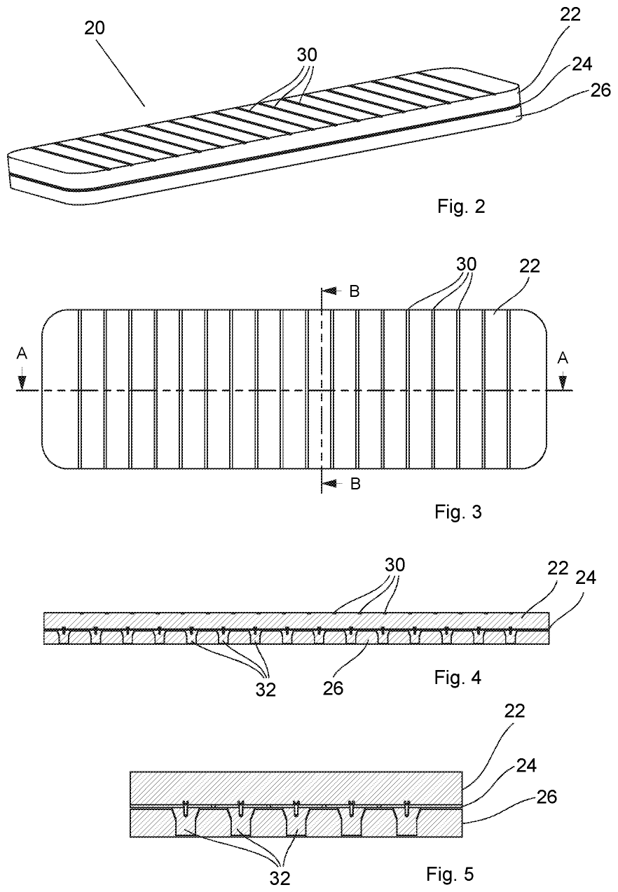

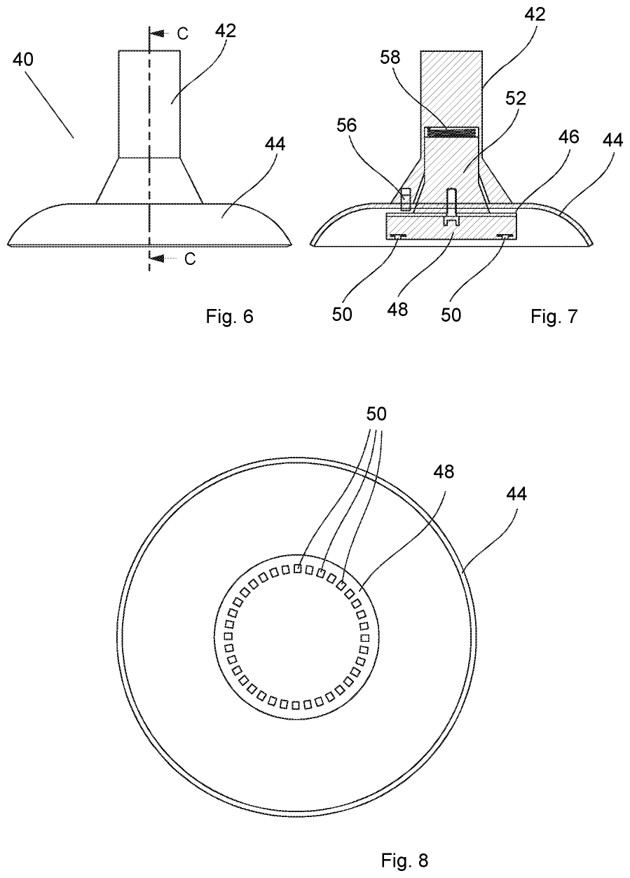

FIG. 1 shows a treatment system according to an embodiment of the present disclosure. It is important to note that the treatment system is for treating a patient by causing changes within the patient's body whereas imaging devices are for creating an image of a patient's body without causing changes in the patient's body. The present disclosure is strictly related to treatment devices and systems. The treatment system comprises a base unit 20 and a portable unit 40. The details of the base unit have been shown in more detail in FIGS. 2-5 and the portable unit in FIGS. 6-8. The treatment system may also include a protective cover 60 protecting both the patient and an operator of the treatment system from unwanted ultraviolet emissions. The portable unit can be attached to the base unit but movable in relation to the base unit.

The base unit 20 (shown in FIGS. 2-5) can be in a form of a mattress or in an embodiment similar to the portable unit 40. The base unit comprises a pliable top layer 22 which can adapt to the shape of the patient placed against the it. The pliable top layer can be made out of silicone, silicone-based material or some other pliable material where ultrasound waves can propagate without significant losses. In an embodiment the top layer can be a plastic bladder or silicone bladder filled with water or gel.

The base unit also comprises one or more ultrasonic transducers 32 configured to produce ultrasound waves into the pliable top layer 22. The frequency of the ultrasound produced by the ultrasonic transducers can be e.g. in a range from 100 kHz to 1200 kHz. The base unit 20 preferably also comprises a solid layer 24 which is preferably in contact with the pliable top layer 22 over a large area of the solid layer 24. The one or more ultrasonic transducers 32 are preferably fixed to the solid layer 24. The solid layer can be e.g. a metal plate, such as an aluminium plate or a steel plate which provides the base unit with rigidity and increases transmission of ultrasound waves from the ultrasonic transducers into the pliable top layer 22 which is on one side of the solid layer. On the opposite side of the solid layer 24 is a bottom layer 26. The ultrasound waves from the ultrasonic transducers 32 are prevented from entering into the bottom layer 26 by using material, such as a foam, in the bottom layer 26 where the ultrasound waves do not propagate properly. There can also be an air gap between the solid layer 24 and the bottom layer 26 to ensure that most of the ultrasonic energy is directed into the pliable top layer.

The portable unit 40 (shown in FIGS. 6-8) is preferably a handheld device as shown in FIG. 1 and thereby it preferably comprises a handle 42. The portable unit can also be attached to a movable arm that can be manually or remotely controlled to move the portable unit in a desired direction. The portable unit 40 comprises a pliable pad 48 which can adapt to the shape of the patient placed against the it. The pliable top layer can be made out of silicone, silicone-based material or some other pliable material where ultrasound waves can propagate without significant losses. In an embodiment the top layer can be a plastic bladder or silicone bladder filled with water or gel.

The portable unit comprises an ultrasonic transducer 52 configured to produce ultrasound waves into the pliable pad 48. The frequency of the ultrasound produced by the ultrasonic transducers can be e.g. in a range from 100 kHz to 1200 kHz. The portable unit 40 preferably also comprises a solid layer 46 which is preferably in contact with the pliable pad 48 over a large area of the solid layer 46. The ultrasonic transducers 52 is preferably fixed to the solid layer 46. The solid layer can be e.g. a metal plate, such as an aluminium plate or a steel plate which increases transmission of ultrasound waves from the ultrasonic transducers into the pliable pad 48.

The treatment system further comprises a source of ionizing electromagnetic radiation 50 which may form a part of the base unit 20, part of the portable unit 40 or the source can be a separate unit from which the ionizing electromagnetic radiation is guided to the patient either directly or through the portable unit or the base unit. The source of ionizing electromagnetic radiation 50 can be, for example, an ultraviolet emitter that emits ultraviolet light, an X-ray tube that emits X-rays or a gamma ray emitter that emits gamma rays. In the following embodiments, the source of ionizing electromagnetic radiation 50 is an ultraviolet emitter but the treatment system of the present disclosure can also be used with said other types of ionizing electromagnetic radiation and the following examples are not limited to use of ultraviolet emitters only.

In an embodiment of the present disclosure, the portable unit 40 further comprises one or more ultraviolet emitters 50 as the source of ionizing electromagnetic radiation 50. Said one or more ultraviolet emitters 50 are configured to emit ultraviolet light in a wavelength range from 100 nm to 280 nm. Preferably, the ultraviolet emitters 50 are light emitting diodes (LEDs) having the highest emission peak at a wavelength range from 250 nm to 270 nm. In an embodiment, the one or more ultraviolet emitters 50 of the portable unit 40 are arranged to emit said ultraviolet light into the pliable pad 48. In this case, the pliable pad is at least partially translucent for emitted wavelengths, for example side edge(s) of the pliable pad 48 may be opaque to prevent the ultraviolet emission from escaping the pliable pad into unwanted directions. The ultraviolet emitters may also be covered with a clear plastic or glass cover for allowing the ultraviolet emission to propagate but at the same time protecting the ultraviolet emitters from direct contact with the patient and the patient from the ultraviolet emitters. The unwanted ultraviolet emission can also be blocked with a dome 44 which can be rigid or resilient dome essentially blocking any gaps between the patient and the side of the portable unit 40 that is placed against the patient.

In an embodiment, the base unit 20 also comprises one or more ultraviolet emitters 30 configured to emit ultraviolet light in a wavelength range from 100 nm to 280 nm. Preferably, also the ultraviolet emitters 30 of the base unit 20 are light emitting diodes (LEDs) having the highest emission peak at a wavelength range from 250 nm to 270 nm. Arrangements similar to the portable unit 40 can be used in the base unit when placing the ultraviolet emitters.

Since excessively large dose of ionizing electromagnetic radiation, such as ultraviolet radiation, is harmful, the dosage is preferably restricted. For example, the ultraviolet emitters 30, 50 of the system can be configured to operate at full power for example at most 100 milliseconds, 1 second or 10 seconds at a time. The restriction can also be a power restriction to certain wattage or an energy-based restriction where a patient can only receive a certain amount of energy in a single treatment.

Power levels of ultrasound and ultraviolet light can vary greatly depending on a body part that is treated with the device. For example, an infection in human fingernail can be treated with relatively low power levels compared to a respiratory infection. Even higher power levels may be needed for treating larger mammals, such as treating a horse suffering from a gastroentiritis. Therefore the power levels of the device are adjustable and should be adjusted case by case by a medical professional. Similarly, frequency of the ultrasound can be adjusted depending on a tissue that is treated to achieve desired penetration of ultrasound in to the tissue.

In an embodiment, where the base unit 20 comprises multiple ultrasonic transducers 32, the system can be configured to activate one or more of these multiple ultrasonic transducers 32 of the base unit 20 upon activation of the ultrasonic transducer 52 of the portable unit 40. The activated ultrasonic transducers can be predetermined according to a treatment plan or a set of ultrasonic transducers can be manually selected to begin transmission once the ultrasonic transducer 52 of the portable unit is activated. The activation of the portable unit can be transmitted wirelessly or by wired connection to the base unit. Alternatively, the base unit can be configured to detect the activation of the portable unit by sensing ultrasound waves with a separate detector (not shown in the drawings) or with one or more ultrasonic transducers 32 of the base unit 20.

The same principle can be applied to the ultraviolet emitters. In an embodiment, where the base unit 20 comprises multiple ultraviolet emitters 30, the system can be configured to activate one or more of the multiple ultraviolet emitters 30 of the base unit 20 upon activation of the one or more ultrasonic emitters 50 of the portable unit 40. The activated ultraviolet emitters can be predetermined according to a treatment plan or a set of ultraviolet emitters can be manually selected to begin emission once one or more of the ultraviolet emitters 50 of the portable unit are activated. The activation of the portable unit can be transmitted wirelessly or by wired connection to the base unit. Alternatively, the base unit can be configured to detect the activation of the portable unit by sensing ultraviolet emission with a separate sensor (not shown in the drawings).

In an embodiment of the present disclosure, the base unit 20 comprises multiple ultrasonic transducers 32 and said ultrasonic transducers 32 are configured to detect ultrasonic transmission of the ultrasonic transducer 52 of the portable unit 40. The treatment system is configured to selectively activate one or more of the ultrasonic transducers 32 of the base unit 20. The activated ultrasonic transducers are preferably the ones that detect the strongest signal from the portable unit 40, or alternatively the ultrasonic transducer that detects the strongest signal is activated as well as neighbouring ultrasonic transducers directly next to the one sensing the strongest signal. Selectively activating means that the number of activated ultrasonic transducers 32 is at least one but smaller than the total amount of ultrasonic transducers 32 of the base unit 20.

These embodiments of the treatment system make it possible to apply ultrasound to patient's body from opposite sides simultaneously. In an embodiment, the same applies to the ultraviolet light. This feature allows for lower power levels to be used while still penetrating the body with ultrasound or ultraviolet light. The inventors have found that the ultrasound and ultraviolet light have a strong synergistic effect on bacteria and viruses. Even the ultrasound alone has proven to be effective to reduce inflammation. The ultrasound weakens capsules of the bacteria and other outermost parts of bacteria and viruses. The weakening allows for the ultraviolet light to damage the bacteria and viruses with significantly less power compared to a case where just ultraviolet light is used. The combination of ultrasound and ultraviolet light reduces the needed light intensity by a factor of at least ten. Therefore, intensity and duration of the ultrasound and the ultraviolet light are preferably adjustable for achieving a desired result of the treatment.

The ultraviolet light, applied immediately or soon after applying the ultrasound, is intended to damage the bacteria or viruses so that reproduction of bacteria and virus replication are fully or essentially prevented. The time between the ultrasound and ultraviolet light should be less than one second and preferably the ultraviolet light is applied immediately when ultrasound is applied. Preferably the ultraviolet light is applied while the ultrasound transmission is still ongoing. In an embodiment, ultrasound is applied continuously and ultraviolet light is applied in pulses lasting less than a second or less than 100 milliseconds while ultrasound is being applied. Many of the most severe symptoms can be avoided when the bacteria cannot reproduce and viruses cannot replicate, because then the patient's immune system will be less likely to overshoot when attacking these bacteria and viruses.

The treatment system may also comprise a safety switch to protect the patient and the operator of the treatment system. The safety switch prevents accidental activation of the ultraviolet emitters of the portable unit or the whole system. In an embodiment, the pliable pad 48 and the metal plate 46 of the portable unit 40 are at rest at a first position (shown in FIG. 7) where the metal plate 46 is not in contact with a pin 56. A galvanic contact between the pin 56 and the metal plate 46 is needed for activating the circuit powering the ultraviolet emitters. Thereby, in the first position the one or more ultraviolet emitters 50 are inoperable. The pliable pad 48 and the metal plate 46 are configured to be movable to a second position where the metal plate 46 is in contact with the pin 56 and thereby the one or more ultraviolet emitters 50 are operable. In an embodiment, the ultraviolet emitters 30 of the base unit 20 are also inoperable in the first position of the portable unit and operable in the second position of the portable unit. The portable unit has preferably a spring-bias towards the first position where e.g. a helical spring 58 exerts a force on the pliable pad and the metal plate which has to be overcome by exerting a higher force in the opposite direction to e.g. the pliable pad. This is typically achieved by pushing the pliable pad 48 of the portable unit against a patient.

It is obvious to the skilled person in the art that, as technology develops, the basic idea of the invention can be implemented in various ways. The invention and its embodiments are therefore not limited to only the examples presented above, rather they may vary within the scope of the claims.

We claim:

1-6. (canceled)

7. A treatment system comprising:
a base unit and a portable unit, the base unit having a pliable top layer and one or more ultrasonic transducers configured to produce ultrasound waves into the pliable top layer, the portable unit having a pliable pad and an ultrasonic transducer configured to produce ultrasound waves into the pliable pad, the treatment system further having a source of ionizing electromagnetic radiation configured to emit ionizing electromagnetic radiation,
the base unit further having a solid layer, and said one or more ultrasonic transducers being fixed to the solid layer,
the solid layer of the base unit being a metal plate.

8-10. (canceled)

11. A treatment system comprising:
a base unit and a portable unit,
the base unit having a pliable top layer and one or more ultrasonic transducers configured to produce ultrasound waves into the pliable top layer,
the portable unit having a pliable pad and an ultrasonic transducer configured to produce ultrasound waves into the pliable pad,
the treatment system further having a source of ionizing electromagnetic radiation configured to emit ionizing electromagnetic radiation,
the source of ionizing electromagnetic radiation being in the portable unit and having one or more ultraviolet emitters configured to emit ultraviolet light in a wavelength range from 100 nm to 280 nm,
the base unit having multiple ultrasonic transducers, and
the treatment system being configured to activate one or more of the multiple ultrasonic transducers of the base unit upon activation of the ultrasonic transducer of the portable unit.

12. A treatment system comprising:
a base unit and a portable unit,
the base unit having a pliable top layer and one or more ultrasonic transducers configured to produce ultrasound waves into the pliable top layer,
the portable unit having a pliable pad and an ultrasonic transducer configured to produce ultrasound waves into the pliable pad,
the treatment system further having a source of ionizing electromagnetic radiation configured to emit ionizing electromagnetic radiation,
the base unit having multiple ultraviolet emitters; and
the treatment system being configured to activate one or more of the multiple ultraviolet emitters of the base unit upon activation of the one or more ultrasonic emitters of the portable unit.

13. A treatment system comprising:
a base unit and a portable unit,
the base unit having a pliable top layer and one or more ultrasonic transducers configured to produce ultrasound waves into the pliable top layer,
the portable unit having a pliable pad and an ultrasonic transducer configured to produce ultrasound waves into the pliable pad,
the treatment system further having a source of ionizing electromagnetic radiation configured to emit ionizing electromagnetic radiation,
the base unit having multiple ultrasonic transducers and the ultrasonic transducers of the base unit being configured to detect ultrasonic transmission of the ultrasonic transducer of the portable unit;
the treatment system being configured to selectively activate one or more of the ultrasonic transducers of the base unit detecting a strongest signal from the portable unit; and
a number of activated ultrasonic transducers being smaller than a total amount of ultrasonic transducers of the base unit.

14. A treatment system comprising:
a base unit and a portable unit,
the base unit having a pliable top layer and one or more ultrasonic transducers configured to produce ultrasound waves into the pliable top layer,
the portable unit having a pliable pad and an ultrasonic transducer configured to produce ultrasound waves into the pliable pad,
the treatment system further having a source of ionizing electromagnetic radiation configured to emit ionizing electromagnetic radiation,
the portable unit further having a solid layer and said ultrasonic transducer being fixed to the solid layer,
the solid layer of the portable unit being a metal plate,
the pliable pad and the metal plate being at rest at a first position where the metal plate is not being in contact with a pin and thereby the one or more ultraviolet emitters being inoperable, and
the pliable pad and the metal plate being configured to be movable to a second position wherein the metal plate is being in contact with the pin (56) and thereby the one or more ultraviolet emitters (50) are being operable.
